(19) United States
(12) Patent Application Publication
(10) Pub. No.: US 2009/0048620 A1
(43) Pub. Date: Feb. 19, 2009

(54) Apparatus and methods for carpal tunnel release

(75) Inventors: Arnold-Peter C. Weiss, Barrington, RI (US); Robert A. Rabiner, Tiverton, RI (US)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: IlluminOss Medical, Inc.

(21) Appl. No.: 11/893,078

(22) Filed: Aug. 14, 2007

(51) Int. Cl. A61B 17/32

(52) U.S. Cl. 606/167; 128/898

(57) ABSTRACT

An apparatus and methods for treating carpal tunnel syndrome are disclosed herein. A surgical knife and guide tool for performing carpal tunnel release surgery includes an elongated handle having a cutting head comprising an upper edge, a bottom edge, and a cutting edge therebetween, wherein the bottom edge comprises at least one glider extending from the bottom edge of the cutting head; and an elongated body having a longitudinal opening comprising a top portion having a first width for accepting the cutting head of the elongated handle and a bottom portion having a second width for accepting the at least one glider extending from the bottom edge of the cutting head, wherein the at least one glider controls longitudinal movement of the elongated handle.

### RELATED APPLICATIONS

None.

### FIELD

The embodiments disclosed herein relate to medical apparatuses for the treatment of carpal tunnel syndrome, and more particularly to medical devices and methods for the guidance of a surgical knife during carpal tunnel release surgery.

### BACKGROUND

Carpal Tunnel Syndrome (CTS) occurs when the median nerve is squeezed as it courses through the anatomic passageway through the wrist, called the carpal tunnel. Surgery is usually the treatment of choice for carpal tunnel syndrome which includes the cutting of the transverse carpal ligament, which releases pressure on the median nerve. Carpal tunnel release surgery is thought to be the most commonly performed surgical procedure in the United States. Currently, there are a variety of procedures for treating CTS, including Open Release, Mini Open Release, Endoscopy, Ultrasound and Percutaneous Balloon Carpal Tunnelplasty.

Prior techniques for surgically releasing the transverse carpal ligament are described in U.S. Pat. No. 6,685,717 entitled “Scalpel system for treating carpal tunnel syndrome;” U.S. Pat. No. 6,179,852 entitled “Carpal Tunnel Device and Method;” U.S. Pat. No. 6,019,774 entitled “Carpal Tunnel Release Apparatus and Method;” U.S. Pat. No. 5,908,433 entitled “Carpal Tunnel Knife;” U.S. Pat. No. 5,908,431 entitled “Carpal Tunnel Surgery Instruments;” U.S. Pat. No. 5,334,214 entitled “Apparatus and Method for Dividing Transverse Carpal Ligament;” U.S. Pat. No. 5,507,800 entitled “Carpal Tunnel Tome and Carpal Tunnel Release Surgery;” U.S. Pat. No. 5,387,222 entitled “Carpal Tunnel Tome and Carpal Tunnel Release Surgery;” U.S. Pat. No. 5,413,580 entitled “Carpal Tunnel Knife;” and U.S. Pat. Application Ser. No. 2004/0054378 entitled “Set of Surgical Instruments for Carpal Tunnel Surgery.”

The prior devices and methods designed for performing carpal tunnel release surgery present problems to the medical professional and the patient. Due to the lack of control of the knife blade as it passes through the patient's wrist, possible adverse effects include neurovascular injuries to the median nerve, the motor branch of the median nerve, the ulnar nerve, and the superficial palmar arch and other surrounding neurovascular structures, resulting in permanent nerve damage or paralysis. Damage to the tendons surrounding the carpal tunnel, including bowstringing, lacerations and partial lacerations of these tendons have also been found.

Thus, there is a need in the art for an apparatus and methods for performing carpal tunnel release surgery with ease of use, greater control of the surgical knife, and safety to minimize injury to adjacent tissues.

### SUMMARY

An apparatus and methods for treating carpal tunnel syndrome are disclosed. According to aspects illustrated herein, there is provided a surgical knife for performing carpal tunnel release surgery comprising: a handle having a forward portion and a rearward portion; a cutting head extending from the forward portion of the handle, the cutting head comprising an upper edge, a bottom edge, and a cutting edge therebetween, wherein the upper edge terminates in a guide finger that projects forwardly past the cutting edge; and at least one glider extending from the bottom edge of the cutting head to control longitudinal movement of the surgical knife.

According to aspects illustrated herein, there is provided a guide tool for performing carpal tunnel release surgery comprising: a body having a proximal end, a distal end, and a longitudinal axis therebetween; and a longitudinal opening that extends into the body, the longitudinal opening comprising a top portion having a first width for accepting a forward portion of a surgical knife and a bottom portion having a second width for accepting at least one glider extending from a bottom edge of the forward portion of the surgical knife.

According to aspects illustrated herein, there is provided a surgical knife and guide tool for performing carpal tunnel release surgery comprising: an elongated handle having a cutting head comprising an upper edge, a bottom edge, and a cutting edge therebetween, wherein the bottom edge comprises at least one glider extending from the bottom edge of the cutting head; and an elongated body having a longitudinal opening comprising a top portion having a first width for accepting the cutting head of the elongated handle and a bottom portion having a second width for accepting the at least one glider extending from the bottom edge of the cutting head, wherein the at least one glider controls longitudinal movement of the elongated handle.

According to aspects illustrated herein, there is provided a method of cutting a transverse carpal ligament, the method comprising introducing a guide tool into a wrist of a patient, the guide tool comprising a body having a proximal end, a distal end, and a longitudinal axis therebetween and a longitudinal opening that extends into the body, the longitudinal opening comprising a top portion having a first width for accepting a cutting head of a surgical knife and a bottom portion having a second width for accepting at least one glider extending from a bottom edge of the cutting head of the surgical knife; positioning the cutting head of the surgical knife within the longitudinal opening of the guide tool such that the at least one glider on the bottom edge of the cutting head rides within a bottom portion of the longitudinal opening of the guide tool; moving the surgical knife forward within the longitudinal opening of the guide tool so the cutting head of the surgical knife cuts the transverse carpal ligament; and releasing the transverse carpal ligament.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A and FIG. 1B show perspective views of surgical knifes of the presently disclosed embodiments. FIG. 1A shows a surgical knife having a plurality of gliders. FIG. 1B shows a surgical knife having a single glider. FIG. 1A-1 is a close-up view of the surgical knife shown in FIG. 1A.

FIG. 2A and FIG. 2B show perspective views of guide tools of the presently disclosed embodiments. FIG. 2A shows a guide tool having a pair of longitudinal openings on an upper side of the guide tool. FIG. 2B shows a perspective sectional view of a guide tool having a single longitudinal opening on an upper side of the guide tool.

FIG. 3A and FIG. 3B show isolated top plan views of surgical knifes of the presently disclosed embodiments. FIG. 3A shows a surgical knife having a pair of gliders. FIG. 3B shows a surgical knife having a single glider.

FIG. 4A, FIG. 4B and FIG. 4C show close-up views of a surgical knife having a pair of gliders of the presently disclosed embodiments. FIG. 4A is a side elevation view showing a cutting head of the surgical knife. FIG. 4B is a front end view showing a cutting head of the surgical knife. FIG. 4C is a side elevation view showing a cutting head of the surgical knife.

FIG. 4D, FIG. 4E and FIG. 4F show close-up views of a surgical knife having a single glider of the presently disclosed embodiments. FIG. 4D is a side elevation view showing a cutting head of the surgical knife. FIG. 4E is a front end view showing a cutting head of the surgical knife. FIG. 4F is a side elevation view showing a cutting head of the surgical knife.

FIG. 5A, FIG. 5B and FIG. 5C show close-up views of a surgical knife having a pair of gliders of the presently disclosed embodiments. FIG. 5A is a side elevation view showing a cutting head of the surgical knife. FIG. 5B is a front end view showing a cutting head of the surgical knife. FIG. 5C is a side elevation view showing a cutting head of the surgical knife.

FIG. 5D, FIG. 5E and FIG. 5F show close-up views of a surgical knife having a single glider of the presently disclosed embodiments. FIG. 5D is a side elevation view showing a cutting head of the surgical knife. FIG. 5E is a front end view showing a cutting head of the surgical knife. FIG. 5F is a side elevation view showing a cutting head of the surgical knife.

FIG. 6A and FIG. 6B show views of a surgical knife in position within a longitudinal opening of a guide tool of the presently disclosed embodiments. FIG. 6A shows a perspective view of the surgical knife as it enters the guide tool. FIG. 6B shows a cross-sectional view of the surgical knife positioned within the guide tool.

FIG. 7A and FIG. 7B show views of a guide tool of the presently disclosed embodiments. FIG. 7A shows a perspective view of a guide tool having a single longitudinal opening on an upper surface of the guide tool. FIG. 7B shows a cross-sectional view of the guide tool taken along line B-B.

FIG. 8A, FIG. 8B and FIG. 8C show views of a surgical knife and guide tool of the presently disclosed embodiments. FIG. 8A shows a close-up view of a surgical knife having a single glider with a round shape. FIG. 8B shows a guide tool having a single longitudinal opening on an upper surface of the guide tool for accepting the surgical knife of FIG. 8A. FIG. 8C shows a cross-sectional view of the guide tool taken along line C-C.

FIG. 9 is a perspective view showing a light source optically engaging a guide tool of the presently disclosed embodiments.

FIG. 10A and FIG. 10B show the method steps for utilizing a guide tool and surgical knife of the presently disclosed embodiments during a carpal tunnel release procedure.

FIG. 10C is a view showing the release of a transverse carpal ligament after use of a surgical knife and guide tool of the presently disclosed embodiments.

While the above-identified drawings set forth presently disclosed embodiments, other embodiments are also contemplated, as noted in the discussion. This disclosure presents illustrative embodiments by way of representation and not limitation. Numerous other modifications and embodiments may be devised by those skilled in the art which fall within the scope and spirit of the principles of the presently disclosed embodiments.

### DETAILED DESCRIPTION

Medical apparatuses and methods for treating Carpal Tunnel Syndrome (CTS) are disclosed herein. The devices disclosed herein include surgical knifes and protective guide tools which provide a high level of control of the surgical knifes and direct visualization of the median nerve (MN) during a carpal tunnel release procedure. The guide tool has a longitudinal opening for accepting a forward portion of the surgical knife. The surgical knife has at least one glider which allows the surgical knife to move securely forward and backward within the longitudinal opening of the guide tool. The glider helps ensure the forward portion of the surgical knife is positioned to cut the median nerve MN. When the surgical knife is secured within the longitudinal opening, lateral movement of the surgical knife is prevented, thus minimizing injury to adjacent tissues.

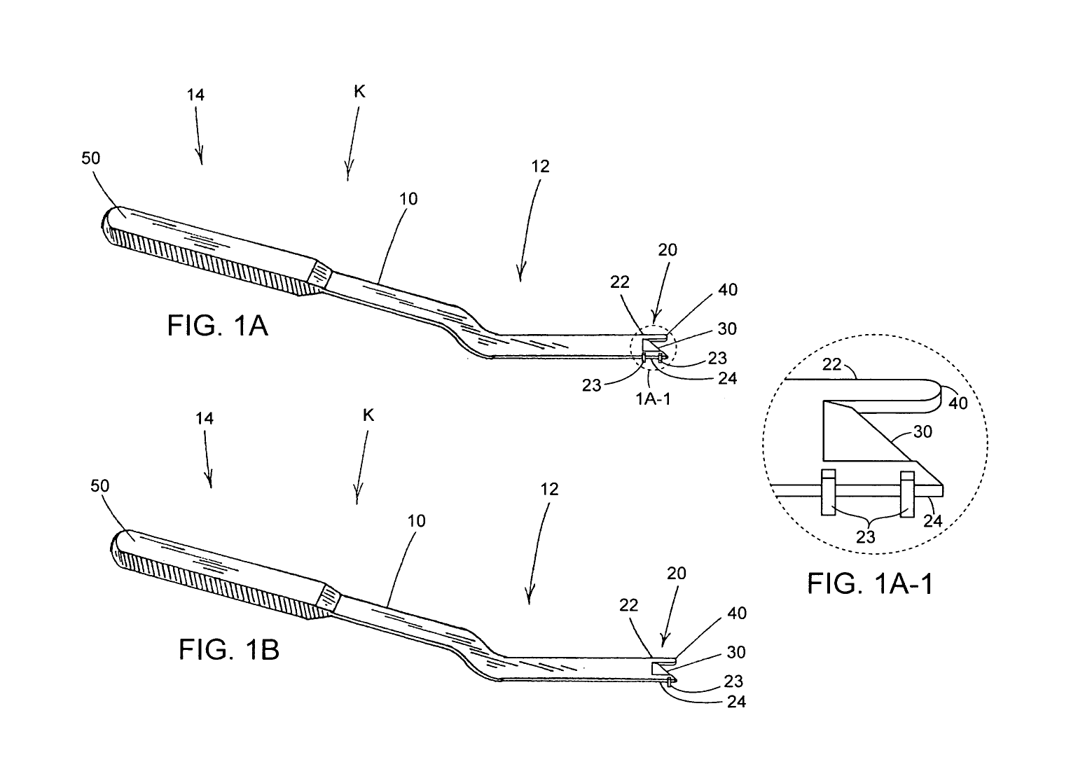

The main components of a surgical knife K for carpal tunnel release surgery are shown generally in FIG. 1A and FIG. 1B. The surgical knife K is designed for performing carpal tunnel release surgery and includes an elongated handle 10 with a forward portion 12 and a rearward portion 14. The forward portion 12 terminates in a cutting head 20, which is shown in FIG. 1A-1. The cutting head 20 defines an upper edge 22 and a bottom edge 24. The bottom edge 24 includes at least one glider 23 for securing the surgical knife K within a longitudinal opening of a guide tool G, thus allowing controlled forward and backward movement of the surgical knife K within the guide tool G. In the embodiment depicted in FIG. 1A, the bottom edge 24 includes a pair of gliders 23. In the embodiment depicted in FIG. 1B, the bottom edge 24 includes a single glider 23. The at least one glider 23 is constructed of any surgically suitable material. In an 1 5 embodiment, the at least one glider 23 is constructed from a metal material. In an embodiment, the at least one glider 23 is constructed from a plastic material. In an embodiment, the at least one glider 23 is machined onto the bottom edge 24 of the cutting head 20 of the surgical knife K.

In an embodiment, the at least one glider 23 is welded onto the bottom edge 24 of the cutting head 20 of the surgical knife K. In an embodiment, the at least one glider 23 is glued onto the 20 bottom edge 24 of the cutting head 20 of the surgical knife K. In an embodiment, the entire surgical knife K including the at least one glider 23, is fabricated as one piece.

The cutting head 20 terminates in a cutting edge 30 which extends from the proximate bottom edge 24 toward the upper edge 22 of the cutting head 20 such that the cutting edge 30 intersects and forms an angle with an upper guide finger 40 which projects forwardly past the 25 cutting edge 30. The positioning of the cutting edge 30 at an angle may help to eliminate sawing motion of the surgical knife K which may lead to loss of control of the surgical knife K.

The rearward portion 14 of the handle 10 may include a grip 50 thereon suitable for gripping the surgical knife K during a carpal tunnel release surgery. The handle 10 may be of any suitable configuration for ease of use during a carpal tunnel release surgery. In an embodiment, 30 the handle is bent or angled between the forward portion and the rearward portion. In an embodiment, the grip on the rearward portion of the handle forms an angle of slightly less than about 180 degrees with the forward portion of the handle. The surgical knife K may be constructed of any surgically suitable material. In an embodiment, the surgical knife is constructed from a disposable material. In an embodiment, the surgical knife is constructed from a metal material. In an embodiment, the surgical knife K is constructed from a carbon material or stainless steel material. In an embodiment, the surgical knife K is constructed from a light transmitting material. In an embodiment, the surgical knife K is constructed from a carbon material or stainless steel material as well as a light transmitting material. In an embodiment, the surgical knife K is fabricated to be disposable, and includes a plastic handle and a metal blade.

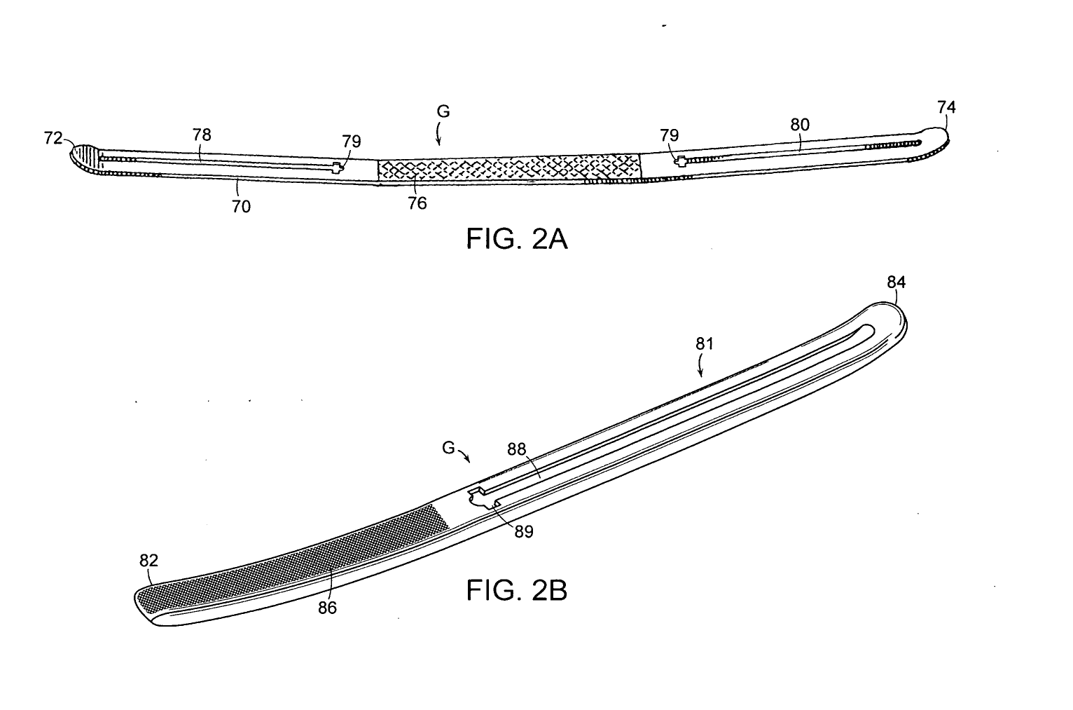

FIG. 2A and FIG. 2B shows various embodiments of guide tools G which may be used in conjunction with one of the surgical knifes K shown in FIG. 1A and FIG. 1B. As shown in FIG. 2A, the guide tool G comprises an elongated bar 70 which is substantially flat on an upper side and terminates in opposite ends 72 and 74 which are preferably slightly curved. In an embodiment, the bar 70 has dimensions that are suitable for performing a carpal tunnel release surgery. The ends 72 and 74 may be different sizes and/or shapes or may be identical in accordance with the presently disclosed embodiments. The bar 70 defines a gripping surface 76 as illustrated in FIG. 2A, and the bar 70 also preferably defines a pair of longitudinal openings 78 and 80, which may be identical or different, positioned on the upper side of the bar 70 on opposite sides of the gripping surface 76.

An entry point 79 at one end of the longitudinal openings 78 and 80 marks the location of the entry and exit for a surgical knife K of the presently disclosed embodiments. Those skilled in the art will recognize that the number of entry points 79 along the length of the longitudinal openings 78 and 80 of the guide tool G may be varied and still be within the scope and spirit of the presently disclosed embodiments. Those skilled in the art will recognize that the placement of the at least one entry point 79 for a surgical knife K may be anywhere along the length of the longitudinal openings 78 and 80 of the guide tool G and still be within the scope and spirit of the presently disclosed embodiments. The entry point 79 may be any shape as long as the shape corresponds to the shape and depth of the at least one glider 23 on the surgical knife K. The entry point 79 will be slightly larger than the at least one glider 23 on the surgical knife K.

The entry point 79 accepts the at least one glider 23 on the bottom edge 24 of the surgical knife K. The surgical knife K engages and disengages the guide tool G at the entry point 79. During the engagement process, the surgical knife K is positioned above the entry point 79 such that the at least one glider 23 of the surgical knife K is aligned with the opening of the entry point 79. The surgical knife K is then brought down so that the at least one glider 23 enters the opening of the entry point 79. Once the at least one glider 23 on the surgical knife K is within the openings of the entry point 79, the surgical knife K is locked into place within the guide tool G and is able to slide along the longitudinal openings 78 and 80. During the disengagement process, the surgical knife K is positioned within the guide tool G such that the cutting head 20 is moved back towards the entry point 79 until the at least one glider 23 on the surgical knife K is positioned within the openings of the entry point 79. The surgical knife K then is able to unlock or disengage from the guide tool G.

FIG. 2B shows an embodiment of a guide tool G of the presently disclosed embodiments. In the embodiment shown in FIG. 2B, the guide tool G comprises an elongated bar 81 which is substantially flat on an upper side and terminates in opposite ends 82 and 84 which are preferably slightly curved. In an embodiment, the bar 81 has dimensions that are suitable for performing a carpal tunnel release surgery. The ends 82 and 84 may be different sizes and/or shapes or may be identical in accordance with the presently disclosed embodiments. The bar 81 defines a gripping surface 86 as illustrated in FIG. 2B of the drawings, and the bar 81 also preferably defines a single longitudinal opening 88, positioned on the upper side of the bar 81. An entry point 89 at one end of the longitudinal opening 88 marks the location of the entry for a surgical knife K of the presently disclosed embodiments.

The longitudinal openings of the guide tools G of the presently disclosed embodiments are positioned on the upper surface and extend into the body of the guide tools G. The longitudinal opening have a top portion having a first width for accepting a forward portion of a surgical knife K and a bottom portion having a second width, the second width is larger than the first width, for accepting an at least one glider that extends horizontally and vertically from a bottom edge of the surgical knife K. In an embodiment, the longitudinal opening is in the shape of an “inverted T.” The at least one glider allows the surgical knife K to be secured and stabilized within the longitudinal opening and enables smooth forward and backward movement of the surgical knife K within the longitudinal opening of the guide tool G. When the surgical knife K is in position within the longitudinal opening, lateral movement of the surgical knife K is minimized, thus minimizing injury to adjacent tissues. When the surgical knife K is in position within the longitudinal opening, the surgical knife K engages the guide tool G to lock the surgical knife K into the guide tool G, thus preventing the surgical knife K from slipping or sliding out of the longitudinal opening of the guide tool G.

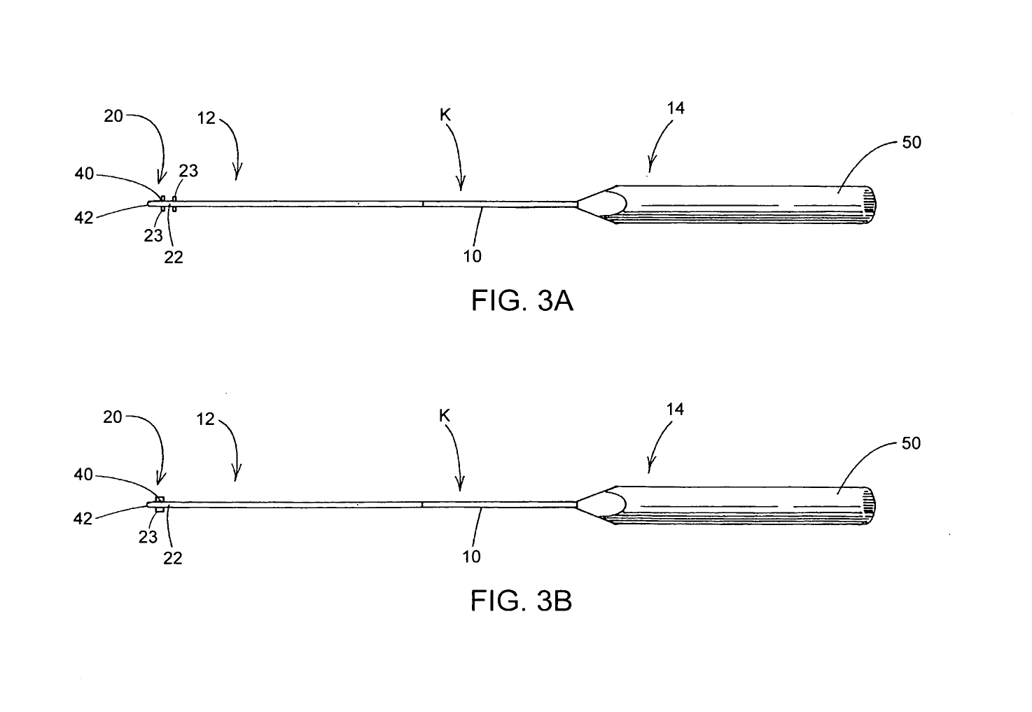

FIG. 3A shows an isolated top plan view of a surgical knife K having a plurality of gliders 23. FIG. 3B shows an isolated top plan view of a surgical knife K having a single glider 23. As shown in FIG. 3A and FIG. 3B, a handle 10 has a forward portion 12 and a rearward portion 14 wherein a grip 50 is positioned on the end of rearward portion 14. As illustrated in FIG. 3A and FIG. 3B, the forward portion 12 of the handle 10 terminates in a cutting head 20 which comprises an upper edge 22 with a guide finger 40. In an embodiment, the guide finger 40 terminates in a rounded terminal end 42. At least one glider 23 extends vertically and horizontally beyond a bottom edge 24 of the cutting head 20. The glider 23 extends horizontally beyond the cutting head 20 to provide stability control. The width of the at least one glider is greater than the width of the surgical knife K. The at least one glider 23 provides stability to the surgical knife K as the surgical knife K moves forward and backward along a longitudinal opening of a guide tool G. The at least one glider 23 locks the surgical knife K within the guide tool G to ensure that the surgical knife K will not slip or slide out of the longitudinal opening of the guide tool G, minimizing damage to surrounding tissues and nerves. The gliders 23 may be any shape including, but not limited to, round, rectangular, square or bars. Those skilled in the art will recognize that the gliders 23 may be of any shape, size and number, and still be within the scope and spirit of the presently disclosed embodiments. In an embodiment, the at least one glider 23 is machined onto the bottom edge 24 of the cutting head 20 of the surgical knife K. In an embodiment, the at least one glider 23 is welded onto the bottom edge 24 of the cutting head 20 of the surgical knife K. In an embodiment, the at least one glider 23 is glued onto the bottom edge 24 of the cutting head 20 of the surgical knife K. In an embodiment, the entire surgical knife K including the at least one glider 23, is fabricated as one piece.

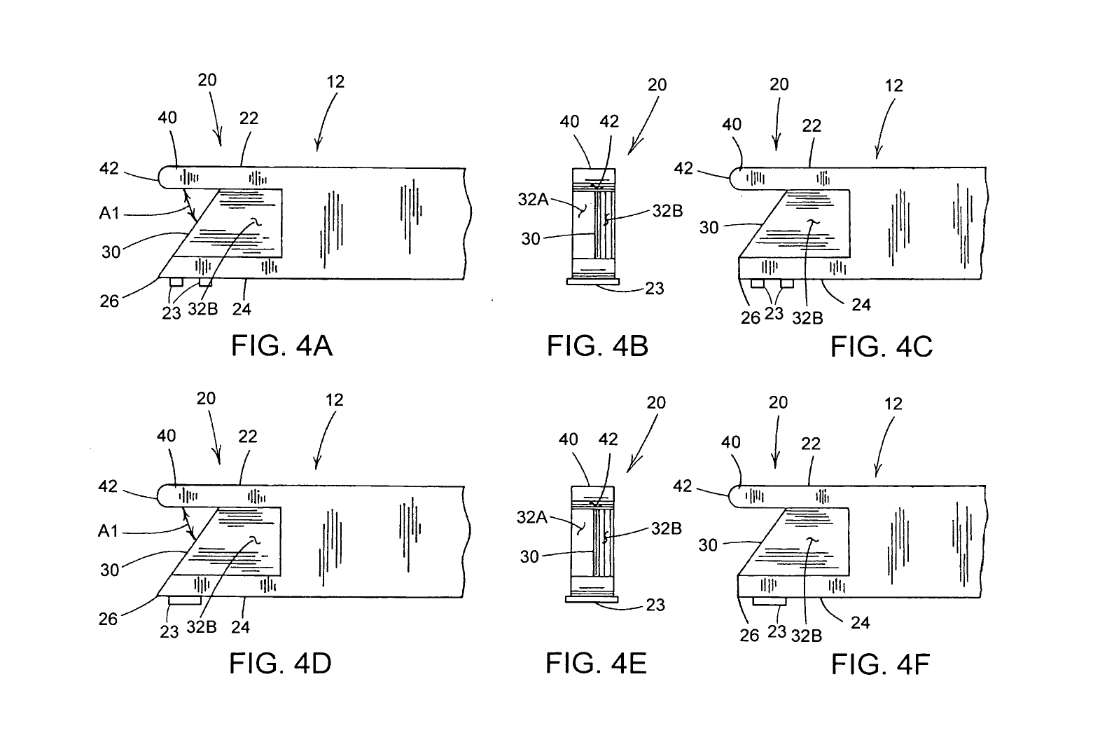

FIG. 4A, FIG. 4B, and FIG. 4C show close-up views of an embodiment of a surgical knife K having a pair of gliders 23 according to the presently disclosed embodiments. FIG. 4D, FIG. 4E, and FIG. 4F show close-up views of an embodiment of a surgical knife K having a single glider 23 according to the presently disclosed embodiments. FIGS. 4A and 4D show side elevation views of a cutting head 20. As shown, the cutting head 20 includes a bottom edge 24 and an upper edge 22 which terminates in a rounded guide finger 40 (which terminates in a rounded terminal end 42). The middle portion of the cutting head 20 terminates in a cutting edge 30 which may be linear and extends from the proximate bottom edge 24 to the proximate upper edge 22.

In an embodiment, the cutting edge 30 is a double-beveled sharp cutting edge resulting from the terminal point of intersection of tapering cutting sides 32A and 32B as shown in FIG. 4B and FIG. 4E. The cutting head 20 is configured such that cutting edge 30 intersects and forms an angle Al with the upper guide finger 40. Angle Al formed between the cutting edge 30 and the guide finger 40 is about 90 degrees or less. In an embodiment, angle Al is about 55 degrees, as shown in FIG. 4A and FIG. 4D. Although it is envisioned according to the presently disclosed embodiments that the cutting edge 30 extend from its intersection with the upper guide finger 40 all the way to the terminal end 26 of the bottom edge 24, it is possible that the cutting edge 30, and the cutting sides 32A and 32B, stop short from the terminal end 26 of the bottom edge 24, as shown in the various figures.

As shown in FIG. 4A and FIG. 4D, the guide finger 40 extends or projects beyond the plane defined by the cutting edge 30. In this embodiment, the rounded terminal end 42 of the upper guide finger 40 and the terminal end 26 of the bottom edge 24 both terminate straight across from one another as both extend distally an identical distance. Alternatively, the terminal end 26 of the bottom edge 24 may stop short of and be behind the plane defined by the cutting edge 30 such as in the configuration shown in FIG. 4C and FIG. 4F of the drawings wherein the terminal end 26 of the bottom edge 24 does not extend all the way to or beyond the plane of the cutting edge 30.

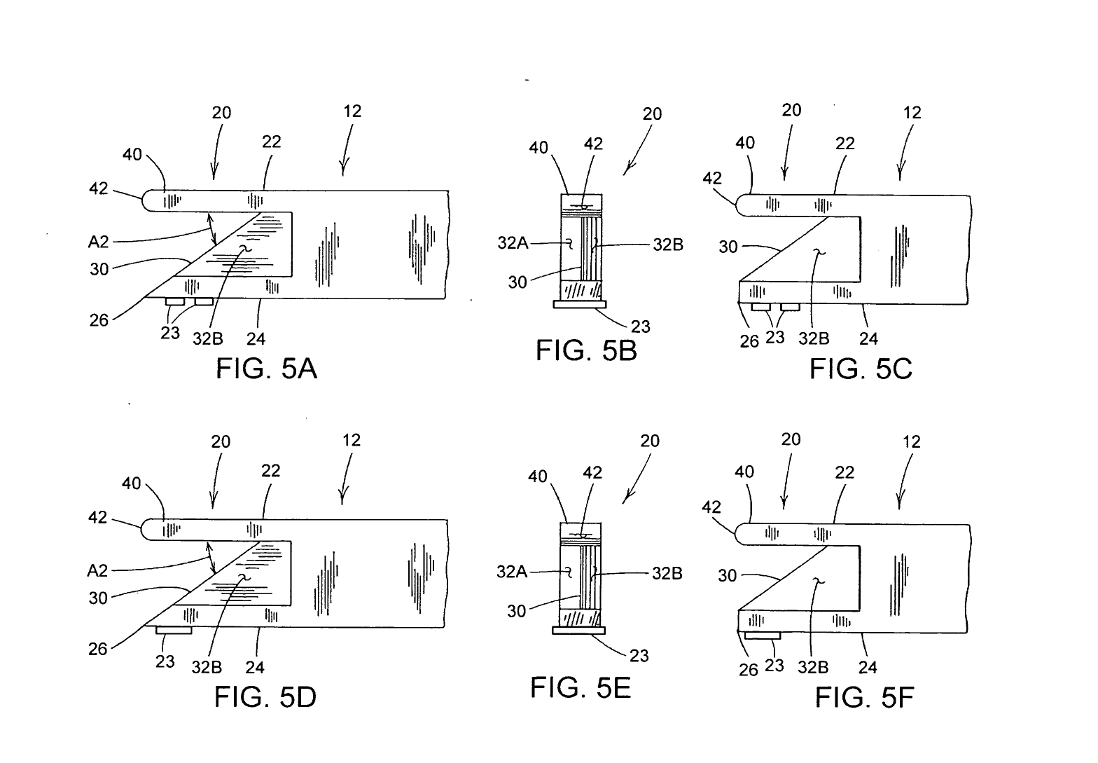

FIG. 5A, FIG. 5B, and FIG. 5C show close-up views of an embodiment of a surgical knife K having a pair of gliders 23 according to the presently disclosed embodiments. FIG. 5D, FIG. 5E, and FIG. 5F show close-up views of an embodiment of a surgical knife K having a single glider 23 according to the presently disclosed embodiments. In the embodiments illustrated in FIG. 5A, FIG. 5B, FIG. 5D and FIG. 5E, the angle formed at the intersection of the upper guide finger 40 and the cutting edge 30, which is designated angle A2, is about 35 degrees. The rounded terminal end 42 of the upper guide finger 40 and the terminal end 26 of the bottom edge 24 continue to both terminate straight across from one another as both extend identical distances distally. FIG. 5C and FIG. 5F show the embodiment of the cutting head 20 shown in FIG. 5A, FIG. 5B, FIG. 5D and FIG. 5E of the drawings with the terminal end 26 of the bottom edge 24 terminating short of and being behind the plane defined by the cutting edge 30 similar to FIG. 4C and FIG. 4F.

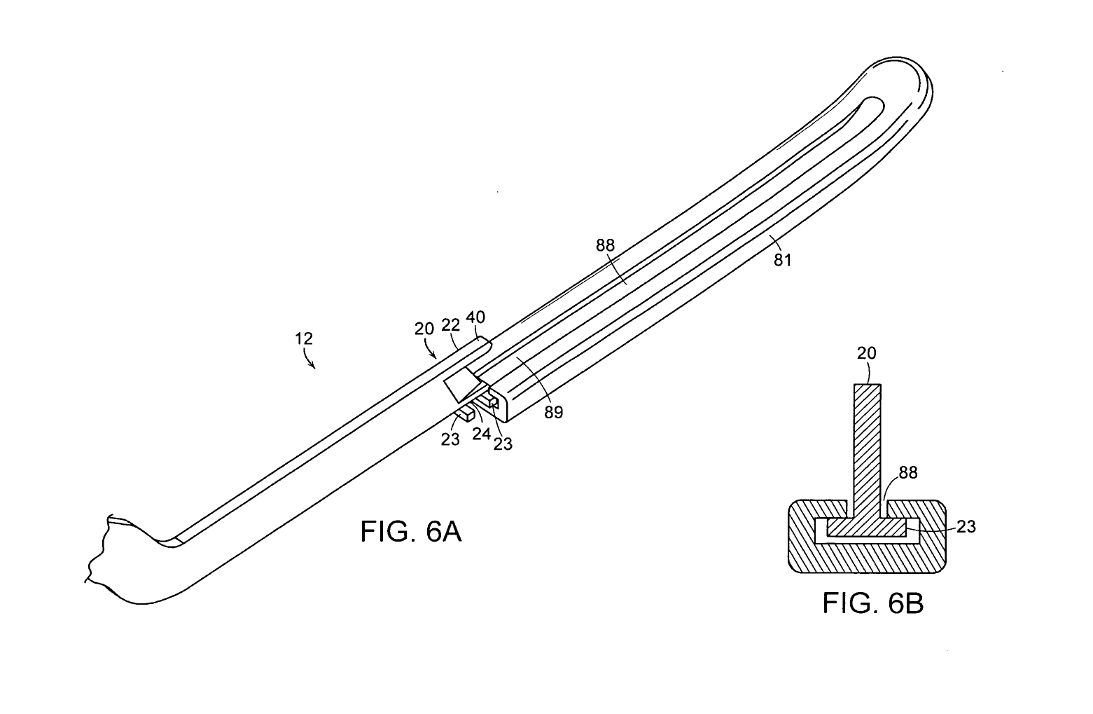

FIG. 6A shows a perspective view of a surgical knife K having a plurality of gliders 23 in position within a guide tool G. Surgical knife K enters the longitudinal opening 88 of guide tool G at entry point 89. In the embodiment depicted in FIG. 6A, the longitudinal opening 88 is in the shape of an “inverted T”. The gliders 23 ride within the arms of the “inverted T” such that the surgical knife K is secured in place within the guide tool G. With the surgical knife K secured in place within the longitudinal opening 88, forward and backward movement of the surgical knife K is performed with ease, while lateral movement of the surgical knife K is minimized. FIG. 6B shows a cross sectional view where the gliders 23 of the surgical knife K are positioned within the arms of the “inverted T” of the opening 88. This positioning locks or secures the surgical knife K in place during use. A user of the medical apparatus is able to perform a carpal tunnel release procedure easily with little or no pressure exerted on the surgical knife K in order to keep the surgical knife K in place during use. The surgical knife K will not slip, slide or otherwise separate out of the guide tool G when the gliders 23 are in the opening 88.

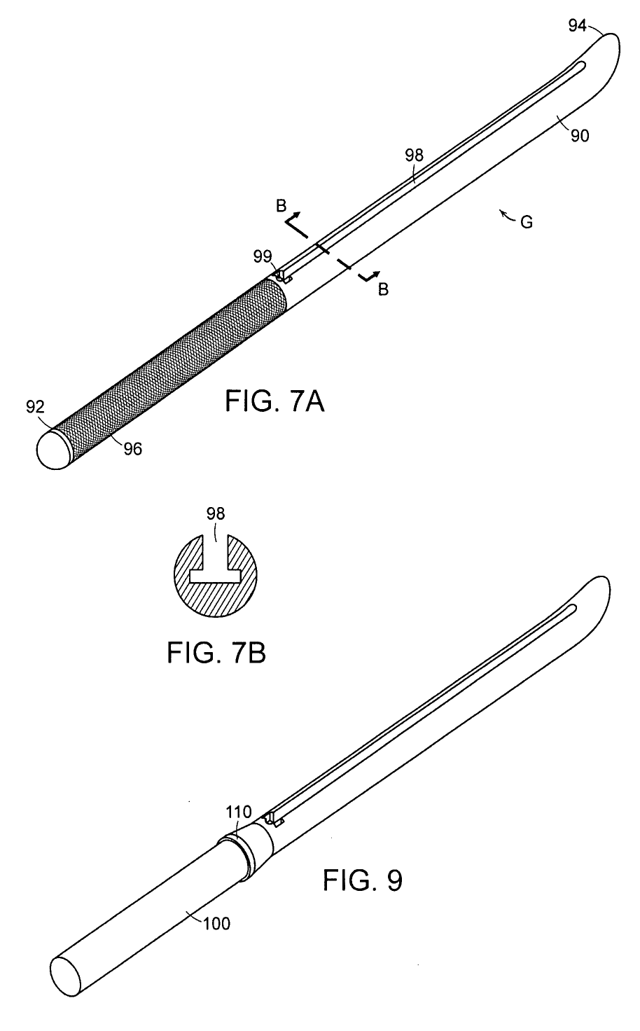

FIG. 7A shows a perspective view of an alternate embodiment of a guide tool G. In this embodiment, the elongated body of the guide tool G is in the shape of a barrel 90 having a proximal end 92, a distal end 94, and a longitudinal axis therebetween. The barrel 90 defines a gripping surface 96 as illustrated in FIG. 7A, and the barrel 90 also preferably defines a single longitudinal opening 98. The overall diameter of the barrel 90 is selected so as to be easily inserted into a carpal tunnel. In an embodiment, the diameter of the barrel 90 is from about 4 millimeters to about 5 millimeters.

An entry point 99 at one end of the longitudinal opening 98 marks the location of the entry for a surgical knife K of the presently disclosed embodiments. Those skilled in the art will recognize that the number of entry points 99 along the length of the longitudinal opening 98 of the guide tool G may be varied and still be within the scope and spirit of the presently disclosed embodiments. Those skilled in the art will recognize that the placement of the at least one entry point 99 for a surgical knife K may be anywhere along the length of the longitudinal opening 98 of the guide tool G and still be within the scope and spirit of the presently disclosed embodiments. The entry point 99 may be any shape as long as the shape corresponds to the shape and depth of the at least one glider 23 on the surgical knife K. The entry point 99 will be slightly larger than the at least one glider 23 on the surgical knife K. A cross-sectional view of the guide tool G taken along line B-B is shown in FIG. 7B, which shows the shape of the longitudinal opening 98.

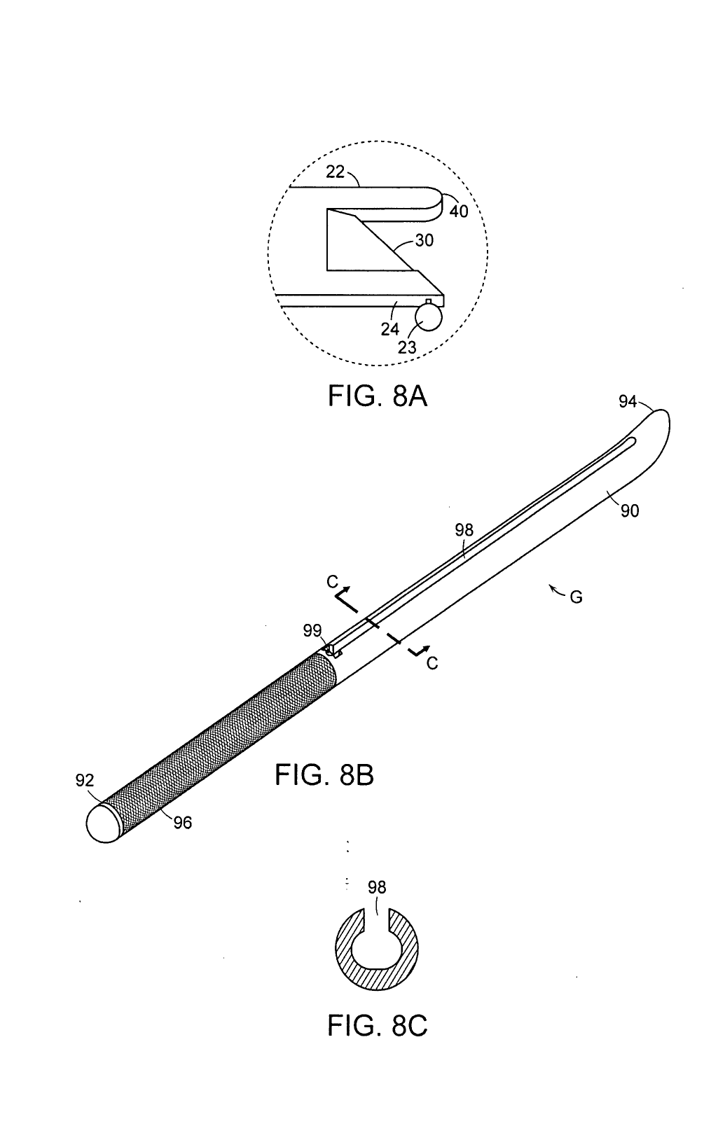

FIG. 8 A shows a perspective view of a forward portion of a surgical knife K terminating in a cutting head 20. The cutting head 20 defines an upper edge 22 and a bottom edge 24. The bottom edge 24 includes at least one glider 23 for securing the surgical knife K within a longitudinal opening of a guide tool G, as shown in FIG. 8B and FIG. 8C, thus allowing controlled forward and backward movement of the surgical knife K within the guide tool G. In the embodiment depicted in FIG. 8A, the bottom edge 24 includes a single glider 23 having a round shape.

FIG. 8B shows a perspective view of an embodiment of a guide tool G for accepting surgical knife K of FIG. 8A. In this embodiment, the elongated body of the guide tool G is in the shape of a barrel 90 having a proximal end 92, a distal end 94, and a longitudinal axis therebetween. The barrel 90 defines a gripping surface 96 as illustrated in FIG. 8B, and the barrel 90 also preferably defines a single longitudinal opening 98. The overall diameter of the barrel 90 is selected so as to be easily inserted into a carpal tunnel. In an embodiment, the diameter of the barrel 90 is from about 4 millimeters to about 5 millimeters. An entry point 99 at one end of the longitudinal opening 98 marks the location of the entry for a surgical knife K of the presently disclosed embodiments. A cross-sectional view of the guide tool G taken along line C-C is shown in FIG. 8C, which shows the shape of the longitudinal opening 98.

The guide tools G of the presently disclosed embodiments may be constructed of any surgically suitable material. In an embodiment, the guide tool G is constructed of a carbon or a stainless steel. Sometimes it may be difficult to locate the exact position of a cutting edge of a surgical knife K while the apparatus is in a patient's hand. A surgeon may locate the apparatus in the patient's hand by feel, mainly by the resistance experienced by the cutting edge and the distance the surgical knife is inserted into the patient's hand. While the surgical knife is inserted, the surgical knife obstructs light entry into the surgical wound thus interfering with the surgeon's ability to see the surgical site. In an embodiment, guide tool G is constructed of a light transmitting material such that the surgical area may be visualized without the use of an added instrument. In an embodiment, the light transmitting material is an optically transparent material including, but not limited to, a glass or a high impact clear plastic such as a polycarbonate or a polyacrylate. In an embodiment, guide tool G is constructed of a carbon material or a stainless steel material and a light transmitting material.

FIG. 9 shows a guide tool G constructed from a light transmitting material. Guide tool G has a connector 110 for connection to a light source 100 for illuminating a surgical site. The light source 100 is bright to provide visualization of the surgical site. The light source 100 is small and non-invasive. The light source 100 engages the guide tool and transmits light through the guide tool G. In an embodiment, the light source 100 is battery-powered. In an embodiment, the light source 100 is a fiber optic cable. In an embodiment, the guide tool G includes a connector for connection to a video screen to enable the viewing of a surgical procedure. The light source 100 may be disposable.

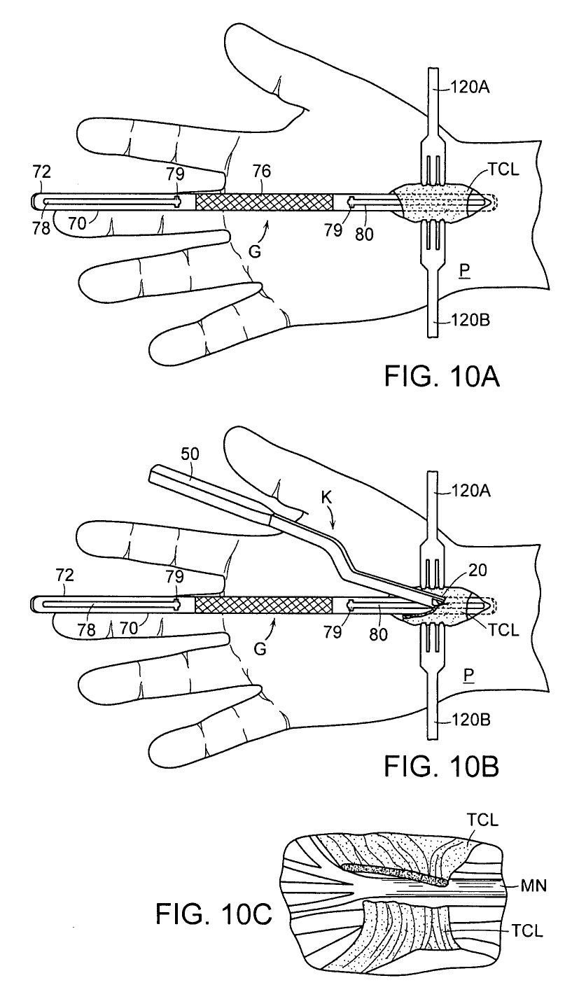

In using the medical apparatuses of the presently disclosed embodiments, access to the carpal tunnel may be accomplished by a medical professional. In an embodiment, a medical professional begins by administering a suitable anesthetic to the palm P of a patient. A typical anesthetic may include about ten cc of anesthetic mixture (five cc of 1% lidocaine without epinephrine plus five cc of 0.25% marocaine without epinephrine). The anesthetic is injected into the midline of the proximal palm P to the proximal wrist crease of the patient. The anesthesia may infiltrate both the carpal tunnel and subcutaneous tissues while being careful not to injure the median nerve (MN). A transverse line is then drawn from the proximal-most extent of the first web space in the palm P of the hand of the patient (designated P in FIG. 10A). A second line is then drawn longitudinally from the radial border of the ring finger proximally. A point about 0.5 to 1 cm proximal to the junction of the transverse line and the second line represents the distal point line of the surgical incision to be made. From the distal point, about 1.5-2 cm surgical incision mark may be drawn in a longitudinal fashion proximally. After tourniquet exsanguination of the upper extremity, a blade of a scalpel is utilized to incise the palmar skin coursing through the palmar fascia to the transverse carpal ligament (TCL) at its distal portion, being careful not to damage the vascular arch. Utilizing either a self-retaining retractor or two Senn retractors, such as those shown as 120A and 120B in FIG. 10A, and one Ragnell retractor, the distal portion of a TCL is identified. The distal portion of the TCL is then incised under direct vision longitudinally as far proximally as possible utilizing a blade, such as No. 15 blade of the scalpel and also utilizing proximal Ragnell retractor for visualization.

The distal end of the guide tool G is then passed underneath the remaining portion of the TCL proximally through the distal incised portion with the top side facing upwardly. The curved blunt tip of the distal end should pass proximately always in contact with the under surface of the TCL until the distal end reaches beyond the proximal extent of the TCL itself. While the guide tool G is maintained in position snugly against the bottom surface of the TCL and after ensuring appropriate passage of the guide tool G beneath the TCL (without tissues between the TCL and the guide tool G itself), the surgical knife K may then be utilized, as shown in FIG. 10B. At least a portion of the cutting head 20 of the surgical knife K is engagedly positioned within the opening 80 of the guide tool G such that the at least one glider located on the bottom edge of the cutting head 20 is secure within the bottom portion of the opening 80. The advancement of the surgical knife K allows the TCL to be strategically positioned for cutting between the guide finger and the guide tool G. Once in this position, the surgical knife K is advanced or passed proximally while the guide tool G is maintained in its position such that the TCL is cut by the cutting edge of the surgical knife K in order to completely release the remaining portion of the TCL. During advancement of the surgical knife K, the portion of the surgical knife K within the opening 80 at least substantially restricts lateral movement thereof and allows the surgical knife K to be suitably guided for cutting only the TCL with minimal risk of accidentally cutting other structures or tissues such as the median nerve MN, as may be appreciated by those of skill in the art. FIG. 10C shows a view of the median nerve MN after the complete release of the TCL using the surgical knife K and the guide tool G of the presently disclosed embodiments. After complete and suitable cutting by the surgical knife K of the TCL, the surgical knife K may then be retracted, and the guide tool G may be utilized to bluntly probe the TCL to ensure its complete release. The wound is then appropriately irrigated and closed. A soft, short palmar dressing may then be placed on the wound, ensuring that full finger and thumb flexion and extension may occur, without difficulty, post-operatively. Patients are encouraged to perform range-of-motion exercises post operatively, although heavy lifting should be avoided. The sutures may typically be removed at 7 to 10 days with progressive increases in hand use counseled for the patient over the ensuing weeks.

A method of cutting a transverse carpal ligament comprises introducing a guide tool into a wrist of a patient, the guide tool having: a body having a proximal end, a distal end, and a longitudinal axis therebetween; and a longitudinal opening that extends into the body, the longitudinal opening comprising a top portion having a first width for accepting a cutting head of a surgical knife and a bottom portion having a second width for accepting at least one glider that extends from a bottom edge of the cutting head; positioning the cutting head of the surgical knife within the longitudinal opening of the guide tool such that the at least one glider on the bottom edge of the cutting head rides within a bottom portion of the longitudinal opening of the guide tool; moving the surgical knife forward within the longitudinal opening of the guide tool such that the cutting head of the surgical knife cuts the transverse carpal ligament; and releasing the transverse carpal ligament.

All patents, patent applications, and published references cited herein are hereby incorporated by reference in their entirety. It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A surgical knife for performing carpal tunnel release surgery comprising:
a handle having a forward portion and a rearward portion;
a cutting head extending from the forward portion of the handle, the cutting head comprising an upper edge, a bottom edge, and a cutting edge therebetween, wherein the upper edge terminates in a guide finger that projects forwardly past the cutting edge; and
at least one glider extending from the bottom edge of the cutting head to control longitudinal movement of the surgical knife.

2. The surgical knife of claim 1 wherein the cutting edge of the cutting head intersects and forms an angle with the guide finger.

3. The surgical knife of claim 2 wherein the angle formed between the cutting edge and the guide finger is about 30 degrees to about 90 degrees.

4. The surgical knife of claim 1 wherein the at least one glider extends horizontally and vertically from the bottom edge.

5. The surgical knife of claim 1 wherein the at least one glider extends beyond a width of the cutting head.

6. The surgical knife of claim 1 wherein a single glider extends from the bottom edge of the cutting head.

7. The surgical knife of claim 1 wherein a pair of gliders extend from the bottom edge of the cutting head.

8. The surgical knife of claim 1 wherein the rearward portion of the handle includes a grip.

9. A guide tool for performing carpal tunnel release surgery comprising:
a body having a proximal end, a distal end, and a longitudinal axis therebetween; and
a longitudinal opening that extends into the body, the longitudinal opening comprising a top portion having a first width for accepting a forward portion of a surgical knife and a bottom portion having a second width for accepting at least one glider extending from a bottom edge of the forward portion of the surgical knife.

10. The guide tool of claim 9 wherein the second width of the longitudinal opening is larger then the first width.

11. The guide tool of claim 9 wherein the longitudinal opening has an inverted T shape.

12. The guide tool of claim 9 wherein the body is in the shape of an elongated bar.

13. The guide tool of claim 9 wherein the body is in the shape of an elongated barrel.

14. The guide tool of claim 9 wherein a single glider extends from the bottom edge of the surgical knife.

15. The guide tool of claim 9 wherein a pair of gliders extend from the bottom edge of the surgical knife.

16. A surgical knife and guide tool for performing carpal tunnel release surgery comprising:
an elongated handle having a cutting head comprising an upper edge, a bottom edge, and a cutting edge therebetween, wherein the bottom edge comprises at least one glider extending from the bottom edge of the cutting head; and
an elongated body having a longitudinal opening comprising a top portion having a first width for accepting the cutting head of the elongated handle and a bottom portion having a second width for accepting the at least one glider extending from the bottom edge of the cutting head, wherein the at least one glider controls longitudinal movement of the elongated handle.

17. A method of cutting a transverse carpal ligament comprising:
introducing a guide tool into a wrist of a patient, the guide tool comprising a body having a proximal end, a distal end, and a longitudinal axis therebetween and a longitudinal opening that extends into the body, the longitudinal opening comprising a top portion having a first width for accepting a cutting head of a surgical knife and a bottom portion having a second width for accepting at least one glider extending from a bottom edge of the cutting head of the surgical knife;
positioning the cutting head of the surgical knife within the longitudinal opening of the guide tool such that the at least one glider on the bottom edge of the cutting head rides within a bottom portion of the longitudinal opening of the guide tool;
moving the surgical knife forward within the longitudinal opening of the guide tool so the cutting head of the surgical knife cuts the transverse carpal ligament; and
releasing the transverse carpal ligament.

18. The method of claim 17 wherein the at least one glider minimizes lateral movement of the surgical knife within the guide tool.

19. The method of claim 17 wherein the at least one glider enables smooth forward and backward movement of the surgical knife within the longitudinal opening of the guide tool.

20. The method of claim 17 further comprising disengaging the surgical knife from the longitudinal opening in the guide tool.
